(19) United States
(12) Patent Application Publication
(10) Pub. No.: US 2008/0086033 A1
(43) Pub. Date: Apr. 10, 2008

(54) Hermetic Bonding

(75) Inventor: Gheorghe Mihalca, North Chelmsford, MA (US)

Correspondence Address: FISH & RICHARDSON P.C.;SMITH & NEPHEW, INC., 150 Minuteman Road, Andover, MA, 01810, US

(73) Assignee: SMITH & NEPHEW, INC., Memphis, TN (US)

(21) Appl. No.: 11/539,160

(22) Filed: Oct. 5, 2006

(51) Int. Cl. A61B 1/06

(52) U.S. Cl. 600/160

(57) ABSTRACT

A device includes a first member including a first material, an optical window, and a second member including a second material dissimilar to the first material, wherein the second member is bonded to the optical window and explosion welded to the first member. An implementation of the device includes an autoclavable medical device including an aluminum body, a stainless steel bridge explosion welded to the body, and a sapphire window bonded to the bridge. A method of manufacturing the device is also disclosed.

### TECHNICAL FIELD

This description relates to hermetic bonding.

### BACKGROUND

Between procedures, multiuse medical devices used in invasive procedures are commonly sterilized by autoclaving, a cleaning method entailing exposure of the device to high-pressure, high-temperature steam during a period of time sufficient to ensure an acceptable level of sterilization. For example, typical autoclaving conditions are specified by the American National Standards Institute (ANSI) and published by the Association for the Advancement of Medical Instrumentation (AAMI) in the ANSI/AAMI ST37-1992 standard. Generally, autoclaving steam temperatures can reach 137° C. and pressures can be greater than 0.2 MPa above atmospheric pressure. Typically, these conditions can be maintained during a period of 30 minutes or more.

A typical medical device undergoes at least about one hundred autoclave cycles during its lifetime. To withstand the repeated cycling, external components of such medical devices are hermetically bonded by, for example, soldering, brazing or conventional welding techniques to provide substantial heat resistance and corrosion resistance.

Soldering is a method of joining base metals using a filler metal having a relatively low melting point (for example, below 450° C.) that is below the melting temperatures of the base metals. The base metals and the filler metal are heated, and the filler metal melts and is drawn into the joint between the base metals.

Brazing is similar to soldering, but utilizes filler metals with higher melting temperatures (for example, above 450° C.) that are below the melting temperatures of the base metals. A variety of metal alloys including, for example, alloys containing copper, silver, tin, zinc, or the like, are commonly used as filler metals in brazing processes.

Welding is a method of joining metals or other materials, such as thermoplastics, by coalescence or fusing of the base materials. In contrast to soldering and brazing processes, conventional welding techniques typically heat the base materials beyond the melting temperature of the base materials to melt the base materials and form a metallurgical bond. In general, the base and filler metals or other materials are heated by a furnace or an induction current, but in some instances are heated by other methods. Explosion welding is a nonconventional solid-state welding process in which a metallurgical bond is formed between two metals under the controlled force of explosives.

### SUMMARY

According to one general aspect, a device includes a first member, an optical window and a second member. The second member is bonded to the window and explosion welded to the first member.

Implementations of this aspect may include one or more of the following features. For example, the first and second members include dissimilar materials. The first member is a housing. The first member is aluminum. The first member includes a main portion and a front portion bonded to one another. The second member is explosion welded to the front portion. The optical window includes a mount and an optical element, and the mount is bonded to the element and to the second member. The mount is brazed or soldered to the element. The mount is welded to the second member. The mount includes stainless steel. The optical element includes sapphire. The second member includes a front body and a bridge member bonded to one another. The front body is bonded to the optical window and the bridge is explosion welded to the first member. The second member is stainless steel. The device has a hermetic life greater than about one hundred autoclave cycles. The first member has a thermal conductivity greater than about twenty-five watts per meter kelvin. The device includes a camera.

According to another general aspect, an autoclavable device for use with an endoscope includes an aluminum body, a stainless-steel bridge explosion welded to the body, and a sapphire window bonded to the bridge.

Implementations of this aspect may include one or more of the following features. For example, the body is a housing. The body includes a main portion and a front portion bonded to one another. The window includes a mount and an optical element, and the mount is bonded to the element and to the bridge. The mount is brazed or soldered to the window. The bridge also includes a front portion bonded to the mount and a bridge member bonded to the front portion. The device has a hermetic life greater than about one hundred autoclave cycles. The device includes a camera.

According to another general aspect, a device includes a first member, a second member explosion welded to the first member, and an optical element bonded to the second member. In addition, the device has a hermetic life greater than one hundred autoclave cycles.

Implementations of this aspect may include one or more of the following features. For example, the device has a hermetic life greater than two hundred fifty autoclave cycles. The device has a hermetic life greater than five hundred autoclave cycles. The device has a hermetic life greater than one thousand autoclave cycles.

According to another general aspect, a device includes a first member having a thermal conductivity greater than twenty-five watts per meter kelvin, a second member coupled to the first member by explosion welding, and a third member brazed or soldered to the second member.

Implementations of this aspect may include one or more of the following features. For example, the first member has a thermal conductivity greater than fifty watts per meter kelvin. The first member has a thermal conductivity greater than one hundred watts per meter kelvin. The third member is an optical member.

According to another general aspect, a device includes a body having a thermal conductivity greater than twenty-five watts per meter kelvin, and an optical element coupled to the body. In addition, the device has a hermetic life greater than one hundred autoclave cycles.

Implementations of this aspect may include one or more of the following features. For example, the body has a thermal conductivity greater than one hundred watts per meter kelvin, and the device a hermetic life greater than two hundred fifty autoclave cycles. The device has a hermetic life greater than five hundred autoclave cycles, and the device a hermetic life greater than five hundred autoclave cycles. The device has a hermetic life greater than one thousand autoclave cycles.

According to another general aspect, an endoscope assembly includes an endoscope and a camera coupled to the endoscope. In addition, the camera includes a body having a thermal conductivity greater than twenty-five watts per meter kelvin, and an optical element bonded to the body. In addition, the device has a hermetic life greater than one hundred autoclave cycles.

Implementations of this aspect may include one or more of the following features. For example, the camera includes a body having a thermal conductivity greater than fifty watts per meter kelvin, and the device a hermetic life greater than two hundred fifty autoclave cycles. The camera includes a body having a thermal conductivity greater than one hundred watts per meter kelvin and the device a hermetic life greater than five hundred autoclave cycles. The device has a hermetic life greater than one thousand autoclave cycles.

According to another general aspect, a method of manufacturing a device having a first member, a second member, and an optical window includes bonding the optical window to the second member and explosion welding the second member to the first member.

Implementations of this aspect may include one or more of the following features. For example, the first and second members are made from dissimilar materials. The optical window includes a mount and an optical element, and the method further includes bonding the mount to the second member and bonding the element to the mount. The step of bonding the optical element to the mount includes brazing or soldering the element to the mount. The step of bonding the mount to the second member includes welding the mount to the second member. The second member includes a front body and a bridge member, and the method further includes explosion welding the bridge member to the first member, bonding the front body to the bridge member and bonding the front body to the optical window. The step of bonding the front body to the bridge member comprises welding, and the step of bonding the front body to the optical window comprises brazing or soldering. The device includes a camera, and the method further includes coupling the camera to an endoscope.

According to another general aspect, a method includes bonding a body composed of a material having a thermal conductivity greater than one hundred watts per meter kelvin to an optical element. In addition, the resulting bond has a hermetic life greater than five hundred autoclave cycles.

According to another general aspect, a method includes coupling a body to a bridge by explosion welding and coupling the bridge to an optical element.

Advantages may include one or more of providing a medical device with an optical window having relatively high thermal conductivity, providing a high-definition video camera for use with an endoscope, providing a relatively high-thermal-conductivity housing with an optical window, providing a hermetic bond between two dissimilar metals that cannot be hermetically bonded by bonding techniques such as soldering, brazing and conventional welding, and cost savings compared to alternative bonding techniques.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates a hermetically-sealed autoclavable device having bonded dissimilar materials.

FIG. 2 illustrates a representative endoscopic system.

FIG. 3 is a perspective view of an autoclavable high-definition video camera head for use with the endoscopic system of FIG. 1.

FIG. 4 is a cross-sectional view of an example implementation of the video camera head of FIG. 3.

FIG. 5 is a cross-sectional view of another example implementation of the video camera head of FIG. 3.

FIG. 6 is a block diagram illustrating an example method or process of forming a video camera housing of a video camera for use with an endoscopic system.

FIG. 7 is a block diagram illustrating another example method of forming a video camera housing of a video camera for use with an endoscopic system.

### DETAILED DESCRIPTION

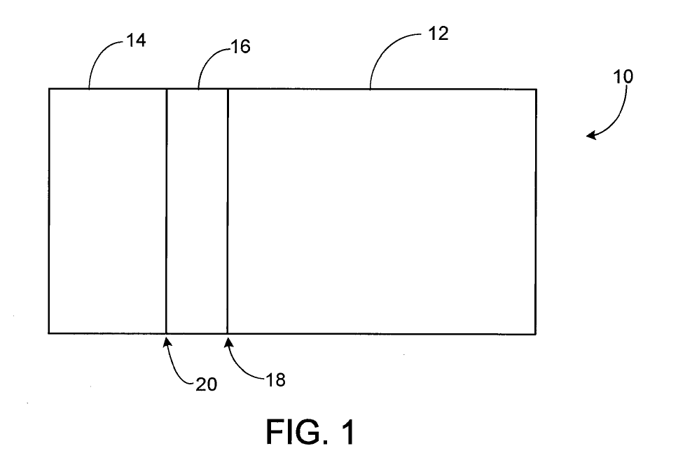

Referring to FIG. 1, a hermetically-sealed autoclavable device 10 includes a first member 12 and a third member 14 formed of dissimilar materials and joined by a second member 16. The material of the first member 12 is selected to have certain properties, for example, light weight and high thermal conductivity, and the material of the third member 14 is selected to have certain properties, for example, optical properties. In order to join the two dissimilar materials, which are incompatible with standard hermetic joining procedures for autoclavable devices that can withstand numerous repeated autoclave cycles, the material of the second member 16 is selected that can be joined to both the first member 12 and the third member 14, for example, by explosion welding at an interface 18 with the first member 12 and laser welding at an interface 20 with the third member 14. Such a construct is capable of withstanding numerous repeated autoclave cycles, for example, greater than about one hundred cycles.

For the purposes of this disclosure, the term “hermetic life” refers to the number of autoclaving cycles to which a device can be exposed while maintaining an acceptable hermetic level, for example, enduring a helium leak test at one atmosphere differential pressure with a leak rate less than or equal to about 1×10⁻⁹ cubic centimeters of helium per second (cc/s He), where an autoclaving cycle is defined as an exposure to saturated steam at a temperature of about 137° C. for a period of about thirty minutes.

Explosion welding—also referred to as explosion bonding or cladding; or explosive welding, bonding or cladding—permits the joining of dissimilar metals, including metals with highly differing properties. Explosion welding is a nonconventional solid-state welding process by which flat metal plates or concentric cylinders are joined by the controlled force of explosives. In contrast to conventional welding methods, the base metals are not melted during explosion welding; instead, a progressive detonation force accelerates one or both of the plates into each other, resulting in a high-velocity, high-interfacial-pressure impact [e.g., about 5,000 to about 7,000 feet per second (ft/s) and about 100 to about 600 thousand pounds per square inch (kpsi)] that forms a durable, high-strength metallurgical bond between the two base metals.

Generally, a layer of explosive material such as a plastic explosive (e.g., ammonium nitrate) is placed over a surface of a cladding plate, which is separated by a small stand-off distance from an adjacent, base plate that is resting on an anvil plate. Then, the explosive material is detonated from one edge of the cladding plate. As the detonation front progresses across the cladding plate, the cladding plate is thrust toward the base plate and a metal surface plasma jet is ejected ahead of the collision front between the two plates, effectively removing oxides and contaminants from the two surfaces, which allows bonding between dissimilar metals that cannot be welded by conventional means. Thus, explosion welding is used to join metals such as aluminum, copper, nickel, alloys containing these metals, stainless steel, Kovar, low-carbon steel, or iron-nickel alloys, or the like to dissimilar metals such as aluminum, copper, nickel, alloys containing these metals, stainless steel, Kovar, iron-nickel alloys, titanium, molybdenum, zirconium, tantalum, niobium, or the like.

In addition, a metal plate can be explosion welded to a dissimilar metal in order to form a bridge that can be used to bond or join additional components that are composed of one or both of the metals, or additional components that are composed of other metals that can be joined to the two metals by conventional means. For example, a stainless steel plate can be explosion welded to an aluminum plate to form a joint, after which an aluminum component can be conventionally welded to the aluminum plate and a stainless steel component can be conventionally welded to the stainless steel plate. Similarly, as another example, a stainless steel plate can be explosion welded to a flat surface of an aluminum component, after which the composite component can be machined and an additional stainless steel component can be conventionally welded to the surface of the stainless steel plate.

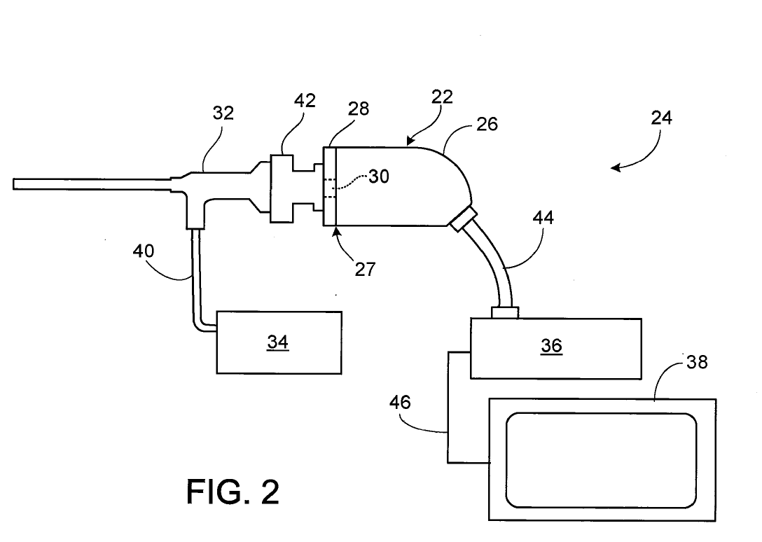

As depicted in FIG. 2, an autoclavable video camera head 22 for use in an endoscopic system 24 includes a first member in the form of a housing 26, a second member in the form of a bridge member, or bridge 28, and a third member in the form of a an optical window 30. In some implementations, the camera head 22 includes a high-definition video camera head, but in other implementations the camera head 22 includes a standard-definition video camera head. The housing 26 is particularly useful in combination with a video camera head 22 that generates heat energy at a relatively high rate compared to some standard video cameras, because the housing 26 is made of, for example, aluminum or an aluminum alloy to provide light weight, high thermal conductivity and corrosion resistance. The optical window 30 includes, for example, an optical sapphire element to provide optical transparency and resistance to steam etching and discoloration. However, because of the difference in thermal expansion properties between aluminum and sapphire, the two materials cannot be directly joined by standard hermetic joining procedures for autoclavable devices. As a result, the bridge 28 is made from a material having thermal expansion properties that are relatively similar to those of sapphire and that is capable of forming a steam resistant joint with the optical window 30, for example, stainless steel, which can be hermetically joined to itself by standard hermetic joining procedures, as well as to sapphire, for example, by way of brazing or soldering.

However, some higher-thermal-conductivity metals and other materials having relatively high thermal conductivity, for example, greater than about 25 W/m K, including aluminum, cannot be welded to stainless steel by conventional welding techniques because of dissimilar properties with respect to stainless steel. For example, the bridge 28 made from stainless steel cannot be welded to the housing 26 made from aluminum using conventional welding. Thus, explosive welding is employed at an interface 27 between the bridge 28 and the housing 26.

The endoscopic system 24 further includes an imaging device in the form of the video camera head 22, or endocamera, to produce a digital electronic image from an optical image of a target subject transmitted by way of an endoscope 32, a light source 34, a digital image processor 36 and a display 38. The light source 34 can be coupled to the endoscope 32 by an optical link 40, for example, a fiber optic cable, to illuminate the target subject under observation beyond the tip of the endoscope 32. In addition, the video camera head 22 can be optically coupled to the endoscope 12 by a coupler 42 to receive an optical image, and electrically coupled to the image processor 36 by an electrical link 44 to transmit a resulting digital image. The image processor 36 can perform signal processing to further refine the digital image, which can then be transmitted by way of another electrical link 46 for display on the display 38, for example, a cathode ray tube (CRT) display or flat panel liquid crystal display (LCD).

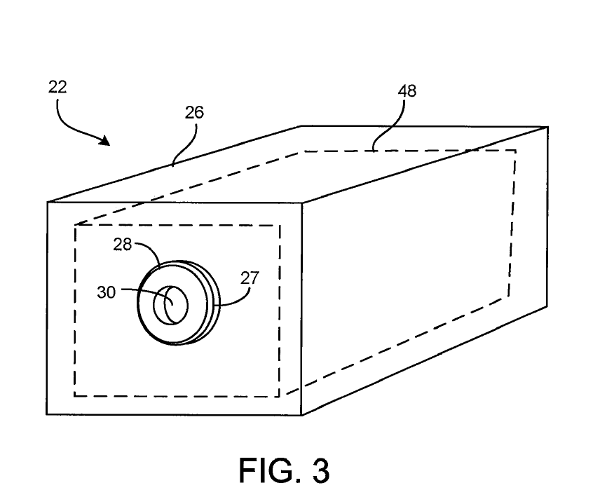

As shown in FIG. 3, the autoclavable video camera head 22 includes a housing 26 enclosing the electronic components 48 of the video camera, which includes one or more sensors to transform an optical image into a digital electronic image, such as three charge-coupled devices (CCDs) configured to image blue, green and red spectra, or other electronic components. In order to allow the optical image from the endoscope 32 to pass into the camera head 22, the optical window 30 is attached to the housing 26 by way of the bridge 28 at the interface 27.

In order to protect the electronic components 48 of the video camera head 22 during autoclaving, the housing 26 is made from materials having substantial resistance to steam corrosion. The housing 26 is hermetically sealed to the bridge 28 and the bridge 28 is hermetically sealed to the window 30 via the methods as mentioned above. The resulting hermetically-sealed video camera head 22 can withstand numerous repeated autoclave cycles, for example, between about one hundred cycles and about one thousand cycles, greater than about one hundred cycles, greater than about two hundred fifty cycles, greater than about five hundred cycles, greater than about one thousand cycles, or more cycles.

Furthermore, in order to accommodate video camera electronic components 48 that generate heat energy at a relatively high rate compared to some standard video cameras, the housing 26 is composed of a material with relatively high thermal conductivity, for example, greater than about 25 Watts per meter kelvin (W/m K), such as aluminum or an aluminum alloy. As a result, the housing 26 dissipates heat at a rate sufficient to maintain an internal ambient at an acceptable working temperature for the electronic components 48. In addition, aluminum or an aluminum alloy provides a relatively light-weight, low-cost housing 26 with favorable machining properties.

Similarly, the optical window 30 not only has acceptable optical characteristics, but also is able to withstand repeated autoclaving cycles. Thus, the optical window 30 includes an optical ceramic material that is highly transparent and resistant to steam etching, such as optical sapphire (monocrystalline aluminum oxide, or alumina, Al₂O₃).

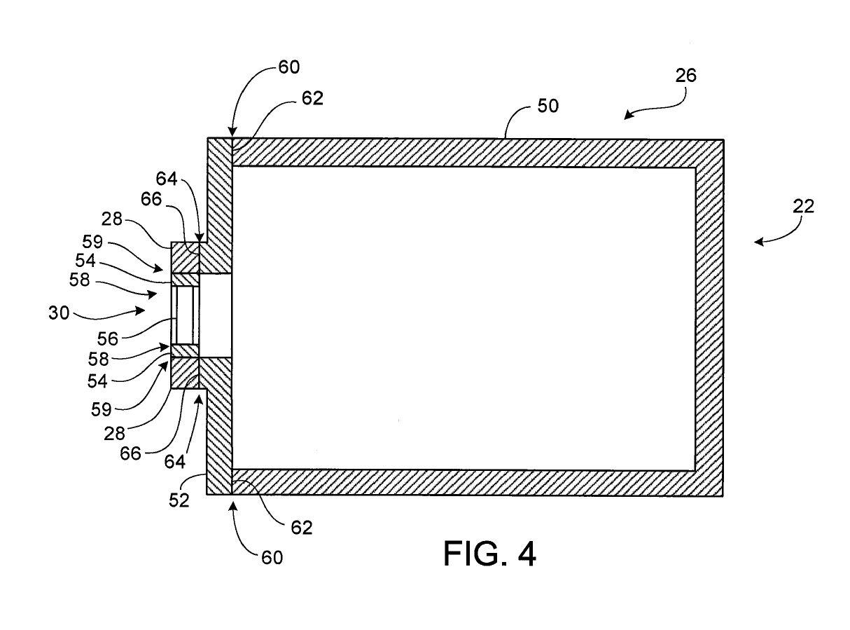

Referring to FIG. 4, an implementation of the autoclavable video camera head 22 includes a housing 26, a bridge 28 and an optical window 30. The housing 26 includes a main body 50 and a front body 52. The optical window 30 includes a window mount 54 and an optical element 56. In this implementation, the main body 50 and the front body 52 are composed of a high-thermal-conductivity metal, for example, greater than about 25 W/m K, such as aluminum or an aluminum alloy; the bridge 28 and the mount 54 are composed of a dissimilar metal such as stainless steel; and the optical element 56 is composed of an optical ceramic material such as sapphire.

A hermetic seal 58 is formed between the optical element 56 and the mount 54 by way of a brazing or soldering process, and a hermetic seal 59 is formed between the mount 54 and the bridge 28 by way of a conventional welding process, such as laser welding. Another hermetic seal 60 is formed at the bond interface 62 coupling the main body 50 and the front body 52 by way of a conventional welding process, such as laser welding. An additional hermetic seal 64 is formed at the bond interface 66 coupling the bridge 28 and the front body 52 by way of explosion welding. In this manner the dissimilar metals comprising the housing 26 and the mount 54 are effectively hermetically joined by way of the bridge 28, facilitating the hermetic joining of the mount 54 to the optical element 56 in order to form the complete hermetically-sealed autoclavable video camera head 22.

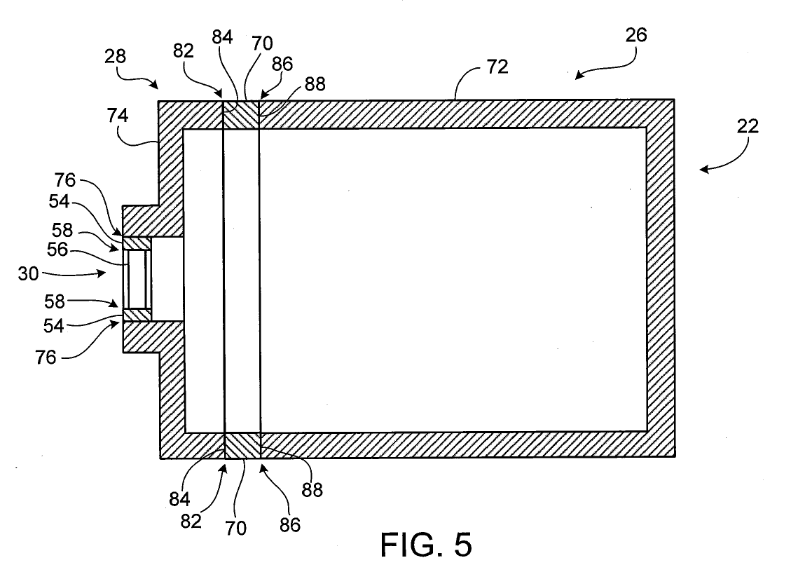

Referring to FIG. 5, another implementation of the autoclavable video camera head 22 includes a housing 26, a bridge 28 and an optical window 30. The housing 26 includes a main body 72 composed of a high-thermal-conductivity metal, for example, greater than about 25 W/m K, such as aluminum or an aluminum alloy. The bridge 28 includes a bridge member 70 and a front body 74 composed of a dissimilar metal such as stainless steel. The optical window 30 includes a window mount 54 composed of the dissimilar material and an optical element 56 composed of an optical ceramic material such as sapphire.

A hermetic seal 58 is formed between the optical element 56 and the mount 54 by way of a brazing or soldering process, and a hermetic seal 76 is formed between the mount 54 and the front body 74 by way of a conventional welding process, such as laser welding. Another hermetic seal 82 is formed at the bond interface 84 coupling the front body 74 and the bridge member 70 by way of a conventional welding process, such as laser welding. An additional hermetic seal 86 is formed at the bond interface 88 coupling the bridge member 70 and the main body 72 by way of explosion welding. In this manner, the metal comprising the bulk of the housing 26 (that is, the main body 72) is effectively hermetically joined to the dissimilar metal of the front body 74 and the mount 54 by way of the bridge 70, facilitating the hermetic joining of the mount 54 to the optical element 56 in order to form the complete hermetically-sealed autoclavable video camera head 22.

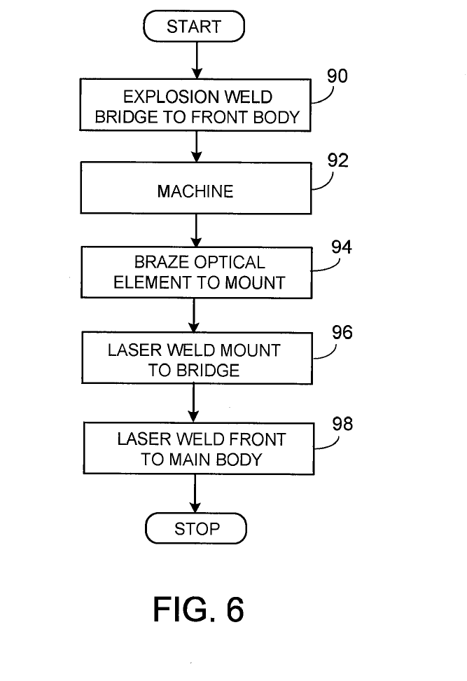

FIG. 6 illustrates an example method of manufacturing a hermetically-sealed autoclavable video camera housing. The process begins in step 90, where a hermetic joint is formed at an interface between two pieces of metal stock in the form of a metal slab and a metal plate to be machined into a bridge member and a front body of the video camera housing, respectively. In this implementation, the slab (front body) is composed of a relatively high-thermal-conductivity metal, for example, greater than about 25 W/m K, such as aluminum or an aluminum alloy, and the plate (bridge member) is composed of a dissimilar metal such as stainless steel.

Then, in step 92, the bridge and the front housing are machined to provide an opening for a window to allow an optical image to pass into the interior of the video camera housing. Next, in step 94, a hermetic joint is formed by way of soldering or brazing at an interface between an optical element, which is composed of an optical ceramic material such as sapphire, and the mount.

Subsequently, in step 96, a hermetic joint is formed by way of a bonding method, such as laser welding, at an interface between the mount and the bridge. Then, in step 98, a hermetic joint is formed by a bonding method, such as laser welding, at an interface between the front body and a main body of the video camera housing, which is also composed of the high-thermal-conductivity metal.

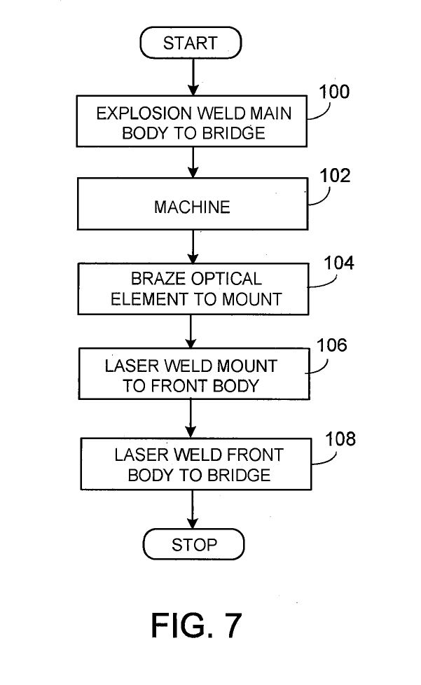

FIG. 7 illustrates another example method of manufacturing a hermetically-sealed autoclavable video camera housing. The process begins in step 100, where a hermetic seal is formed by explosion welding at an interface between two pieces of metal stock in the form of a metal block and a metal plate to be machined into a main body of the video camera housing and a bridge member, respectively. In this implementation, the block (main body) is composed of a relatively high-thermal-conductivity metal, for example, greater than about 25 W/m K, such as aluminum or an aluminum alloy, and the plate (bridge, member) is composed of a dissimilar metal such as stainless steel. Thus, the bridge member serves as an attachment surface for the joining of an additional component of the dissimilar metal.

Then, in step 102, the main body and the bridge are machined to provide a partial housing of the video camera housing. Next, in step 104, the optical element, which is composed of sapphire, is brazed to the mount, which is composed of the dissimilar metal, such as stainless steel. Subsequently, in step 106, a hermetic seal is formed by means such as laser welding at an interface between a window mount and the front body, which are composed of the dissimilar metal. In this implementation, the front housing has been previously machined in a preliminary process to provide an opening for a window to allow an optical image to pass into the interior of the video camera housing. Next, in step 108, a hermetic joint is formed by a bonding method, such as laser welding, at an interface between the front body of the housing and the bridge member.

Additionally, alternative implementations of the autoclavable video camera head and method of manufacture include an optical element composed of any suitable natural or synthetic optical ceramic material, for example, sapphire, spinel (magnesium aluminum oxide, MgAl₂O₄), nitrogen-stabilized aluminum oxide (aluminum oxynitride or ALON™, Al₂₃O₂₇N₅), or the like. Moreover, alternative implementations include a housing partially or wholly composed of any suitable material having relatively high thermal conductivity, for example between about 25 W/m K and about 100 W/m K, greater than about 25 W/m K, greater than about 50 W/m K, greater than about 100 W/m K, or more. Examples of materials having relatively high thermal conductivity include aluminum (Al), copper (Cu), silver (Ag), gold (Au) and alloys of these metals, such as bronze, phosphor bronze, brass, constantan and 2024-T6, 6061 and 4047 aluminum alloys.

It will be understood that various modifications may be made. For example, useful results still could be achieved if steps of the disclosed techniques were performed in a different order and/or if components in the disclosed systems were combined in a different manner and/or replaced or supplemented by other components. Accordingly, other implementations are within the scope of the following claims.

What is claimed is:

1. A device, comprising:
a first member including a first material;
an optical window; and
a second member including a second material dissimilar to the first material, the second member bonded to the optical window and explosion welded to the first member.

2. The device of claim 1, wherein the first member comprises a housing.

3. The device of claim 1, wherein the first member comprises aluminum.

4. The device of claim 1, wherein the first member includes a main portion and a front portion bonded to one another.

5. The device of claim 4, wherein the second member is explosion welded to the front portion.

6. The device of claim 1, wherein the optical window includes a mount and an optical element, the mount being bonded to the element and to the second member.

7. The device of claim 6, wherein the mount is brazed or soldered to the element.

8. The device of claim 6, wherein the mount is welded to the second member.

9. The device of claim 6, wherein the mount comprises stainless steel.

10. The device of claim 6, wherein the optical element comprises sapphire.

11. The device of claim 1, wherein the second member includes a front body and a bridge member bonded to one another.

12. The device of claim 11, wherein the front body is bonded to the optical window and the bridge is explosion welded to the first member.

13. The device of claim 11, wherein the device further comprises a camera.

14. The device of claim 1, wherein the second member comprises stainless steel.

15. The device of claim 1, wherein the device has a hermetic life greater than about one hundred autoclave cycles.

16. The device of claim 1, wherein the first member has a thermal conductivity greater than about twenty-five watts per meter kelvin.

17. The device of claim 1, wherein the device further comprises a camera.

18. An autoclavable device for use with an endoscope, comprising:
an aluminum body;
a stainless-steel bridge explosion welded to the body; and
a sapphire window bonded to the bridge.

19. The device of claim 18, wherein the body comprises a housing.

20. The device of claim 18, wherein the body includes a main portion and a front portion bonded to one another.

21. The device of claim 18, wherein the window includes a mount and an optical element, the mount being bonded to the element and to the bridge.

22. The device of claim 21, wherein the mount is brazed or soldered to the window.

23. The device of claim 21, wherein the bridge further includes a front portion bonded to the mount and a bridge member bonded to the front portion.

24. The device of claim 18, wherein the device has a hermetic life greater than about one hundred autoclave cycles.

25. The device of claim 18, wherein the device further comprises a camera.

26. A device, comprising:
a body having a thermal conductivity greater than twenty-five watts per meter kelvin; and
an optical element coupled to the body, wherein the device has a hermetic life greater than about one hundred autoclave cycles.

27. A method of manufacturing a device having a first member of a first material, a second member of a second material dissimilar to the first material, and an optical window, comprising:
explosion welding the second member to the first member; and
bonding the optical window to the second member.

28. The method of claim 27, wherein the optical window includes a mount and an optical element, the method further comprising:
bonding the optical element to the mount; and
bonding the mount to the second member.

29. The method of claim 28, wherein bonding the optical element to the mount comprises brazing or soldering.

30. The method of claim 28, wherein bonding the mount to the second member further comprises welding.

31. The method of claim 27, wherein the second member includes a front body and a bridge member, the method further comprising:
explosion welding the bridge member to the first member;
bonding the front body to the bridge member; and
bonding the front body to the optical window.

32. The method of claim 31, wherein bonding the front body to the bridge member comprises welding and bonding the front body to the optical window comprises brazing or soldering.

33. The method of claim 27, wherein the device comprises a camera, the method further comprising coupling the camera to an endoscope.
